(19) United States
(12) Patent Application Publication
(10) Pub. No.: US 2007/0173857 A1
(43) Pub. Date: Jul. 26, 2007

(54) Surgical tool and method employing vacuum coupling or magnetic coupling engagement mechanism for facilitating positioning of an intervertebral device

(75) Inventors: Hai H. Trieu, Cardova, TN (US); Jason J. Eckhardt, Memphis, TN (US)

Correspondence Address: HESLIN ROTHENBERG FARLEY & MESITI P.C., 5 COLUMBIA CIRCLE, ALBANY, NY, 12203, US

(73) Assignee: SDGI Holdings, Inc., Wilmington, DE (US)

(21) Appl. No.: 11/340,106

(22) Filed: Jan. 26, 2006

(51) Int. Cl. A61F 5/00; A61F 2/00

(52) U.S. Cl. 606/99

(57) ABSTRACT

A surgical tool and method are provided for positioning an intervertebral device between vertebral members of a patient. The tool includes an insertion/extractoin mechanism including an elongate inserter and an engagement mechanism. The elongate inserter has proximal and distal ends, with the engagement mechanism being disposed at the distal end of the elongate inserter. The engagement mechanism includes an engagement surface configured to at least partially conform to an intervertebral device. The engagement mechanism is adapted to releasably engage the intervertebral device employing one of a vacuum coupling or a magnetic coupling between the engagement surface of the engagement mechanism and the intervertebral device.

### CROSS-REFERENCE TO RELATED APPLICATION

This application contains subject matter which is related to the subject matter of the following application, which is assigned to the same assignee as this application and which is hereby incorporated herein by reference in its entirety:

“Non-Circular Stabilization Sphere and Method”, Allard et al., Ser. No. 11/098,167, filed Apr. 4, 2005 (Attorney Docket No.: P21521.00).

### TECHNICAL FIELD

The present invention relates generally to the field of surgery and medical implants, and more particularly, to surgical tools and methods for positioning an intervertebral device between vertebral members of a patient.

### BACKGROUND OF THE INVENTION

The human spine is a biomechanical structure with thirty-three vertebral members, and is responsible for protecting the spinal cord, nerve roots and internal organs of the thorax and abdomen. The spine also provides structure support for the body while permitting flexibility of motion. A significant portion of the population will experience back pain at some point in their lives resulting from a spinal condition. The pain may range from general discomfort to disabling pain that immobilizes the individual. Back pain may result from a trauma to the spine, be caused by the natural aging process, or may be the result of a degenerative disease or condition.

Procedures to remedy back problems sometimes require correcting the distance between vertebral members by inserting an intervertebral device or spacer between the members. The spacer, which is carefully positioned within the disc space and aligned relative to the vertebral members, is sized to position the vertebral members in a manner to alleviate the patient's back pain.

The intervertebral device may be designed to facilitate insertion into the body. The shape and size of the device are selected to minimize intrusion to a patient during insertion, but still be effective post-insertion to alleviate the pain and provide maximum mobility to the patient.

### SUMMARY OF THE INVENTION

The present invention comprises an insertion tool and method for facilitating positioning an intervertebral device (e.g., a spacer) within a patient. The tools and methods disclosed herein employ tool-to-device attachment approaches that are of sufficient strength for the insertion tool to be readily utilized to accurately insert and place the device (as well as extract the device when necessary). Further, after insertion, the attachment approaches presented provide for detachment and removal of the insertion tool while the intervertebral device remains within the body, and do not deter from the functionality of the device once within the body.

More particularly, in one aspect, presented herein is a surgical tool for inserting/extracting an intervertebral device. The surgical tool includes an insertion/extraction mechanism including an elongate inserter and an engagement mechanism. The elongate inserter has proximal and distal ends, with the engagement mechanism being disposed at the distal end of the elongate inserter. The engagement mechanism includes an engagement surface configured to at least partially conform to the intervertebral device. The engagement mechanism is adapted to releasably engage the intervertebral device employing a vacuum coupling or a magnetic coupling between the engagement mechanism and the intervertebral device.

In another aspect, a surgical tool is provided for inserting/extracting an intervertebral device. The surgical tool includes an insertion/extraction mechanism comprising an elongate inserter and an engagement mechanism. The elongate inserter has proximal and distal ends, with the engagement mechanism being disposed at the distal end of the elongate inserter. The engagement mechanism includes an engagement surface configured to at least partially conform to an intervertebral device. The surgical tool further includes a controller for controlling application of a coupling force between the engagement mechanism and the intervertebral device to releasably engage the intervertebral device when disposed adjacent to the engagement surface for facilitating inserting of the intervertebral device between vertebral members of a patient or extracting of the intervertebral device from between vertebral members of a patient.

In a further aspect, a method of positioning an intervertebral device between vertebral members of a patient is provided. The method includes employing a surgical tool having: an insertion/extraction mechanism comprising an elongate inserter and an engagement mechanism, the elongate inserter having proximal and distal ends, and the engagement mechanism being disposed at the distal end of the elongate inserter, wherein the engagement mechanism includes an engagement surface configured to at least partially conform to an intervertebral device; and wherein the engagement mechanism is adapted to releasably engage the intervertebral device employing a vacuum coupling or a magnetic coupling. The employing of the surgical tool includes: employing the engagement mechanism at the distal end of the elongate inserter to releasably engage an intervertebral device using one of the vacuum coupling or magnetic coupling; inserting the intervertebral device between vertebral members of a patient employing the surgical tool; and detaching the intervertebral device from the engagement mechanism by disengaging the one of the vacuum coupling or magnetic coupling therebetween.

In a still further aspect, a method of withdrawing an intervertebral device from between vertebral members of a patient is provided. The method includes employing a surgical tool having: an insertion/extraction mechanism comprising an elongate inserter and an engagement mechanism, the elongate inserter having proximal and distal ends, and the engagement mechanism being disposed at the distal end of the elongate inserter, wherein the engagement mechanism includes an engagement surface configured to at least partially conform to an intervertebral device; and wherein the engagement mechanism is adapted to releasably engage the intervertebral device employing a vacuum coupling or a magnetic coupling. The employing of the surgical tool includes: inserting the engagement mechanism of the surgical tool into the patient to physically contact the intervertebral device; controllably activating the engagement mechanism to releasably engage the intervertebral device using one of vacuum coupling or magnetic coupling; and withdrawing the engagement mechanism and the intervertebral device from the patient and, thereafter, detaching the intervertebral device from the engagement mechanism by disengaging the vacuum coupling or magnetic coupling therebetween.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a side view of one embodiment of an intervertebral device positioned between two vertebral members, in accordance with an aspect of the present invention;

FIG. 2 is a schematic view of one embodiment of a surgical tool adapted to releasably engage an intervertebral device employing vacuum coupling, in accordance with an aspect of the present invention;

FIG. 2A is a partial schematic view of another embodiment of a surgical tool releasably engaging an intervertebral device employing vacuum coupling, in accordance with an aspect of the present invention;

FIG. 3 is a partial schematic view of still another embodiment of a surgical tool releasably engaging an intervertebral device employing vacuum coupling, in accordance with an aspect of the present invention;

FIG. 4 is a partial schematic view of yet another embodiment of a surgical tool releasably engaging an intervertebral device employing vacuum coupling, in accordance with an aspect of the present invention;

FIG. 5 is a partial schematic view of a further embodiment of a surgical tool releasably engaging an intervertebral device employing vacuum coupling, in accordance with an aspect of the present invention;

FIG. 6 is a schematic depiction of one embodiment of a surgical tool releasably engaging an intervertebral device employing magnetic coupling, in accordance with an aspect of the present invention;

FIG. 7 is a schematic view of a further embodiment of a surgical tool releasably engaging an intervertebral device employing magnetic coupling, in accordance with an aspect of the present invention; and

FIG. 8 is a schematic view of the surgical tool of FIG. 7, showing the pusher shaft in extended position to release the intervertebral device from the surgical tool, in accordance with an aspect of the present invention.

### BEST MODE FOR CARRYING OUT THE INVENTION

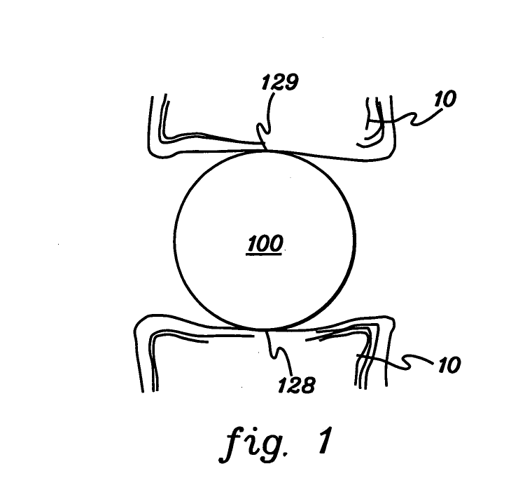

Generally stated, disclosed herein are various surgical inserter tools and methods for facilitating positioning of an intervertebral device in the intervertebral space between vertebral members of a patient. By way of example, the intervertebral device may be a spacer having any one of various oblong, spherical, curved non-spherical, etc. shapes. One non-spherical-shaped embodiment of a spacer is disclosed in the above-incorporated patent application entitled “Non-Circular Stabilization Sphere and Method”. FIG. 1 depicts another spacer embodiment, generally denoted 100, shown positioned between adjacent vertebral members 10 of a patient. In this embodiment, spacer 100 is shown spherical-shaped, with inferior 128 and superior 129 surfaces in contact with the respective vertebral members 10. By way of example only, the exterior surface of spacer 100 is shown as a substantially solid surface.

Those skilled in the art will note that the term “vertebral member” is used generally herein to describe the vertebral geometry comprising the vertebral body, pedicles, lamina, and processes. Likewise, the term “intervertebral space” is used generally to describe the space between vertebral members. The intervertebral space may be formed between adjacent vertebral members, or between non-adjacent vertebral members. The intervertebral device (e.g., spacer 100) may be sized and shaped, and have adequate strength requirements to be used within the different regions of the spine. Although the intervertebral device illustrated and described above is particularly useful in treating the lumbar region of the spine, it should nevertheless be understood that the device is also applicable to other portions of the spine, including the cervical, thoracic, and sacro-iliac regions.

The term “intervertebral device” (e.g., spacer 100) is used herein in a general sense to describe a device that is positioned between vertebral members. In one embodiment, the device is an implant that remains within the body. In another embodiment, the device is a jib which is a fixture or device to guide or hold a cutting, measuring, or space maintaining device in order to prepare a location, such as a vertebral member or intervertebral space, in order to receive an implant. In these embodiments, the device may be removed from the body at the completion of the procedure. By way of example, the intervertebral device may be solid, or have a hollow interior, or even comprise a mesh structure (i.e., when the surgical tool embodiments employing magnetic coupling are utilized).

The terms “upper”, “lower”, “inner”, “outer”, and the like are terms to describe the relative positioning of different elements, and are used in a general sense. The surgical tool and method embodiments described herein are considered in all respects as illustrative and not restrictive, and all changes coming within the meaning and equivalency range of the appended claims are intended to be embraced therein. Although described hereinbelow principally with reference to inserting an intervertebral device in the intervertebral space between vertebral members of a patient, the surgical tools described could alternatively be employed in extracting an intervertebral device from the intervertebral space between vertebral members.

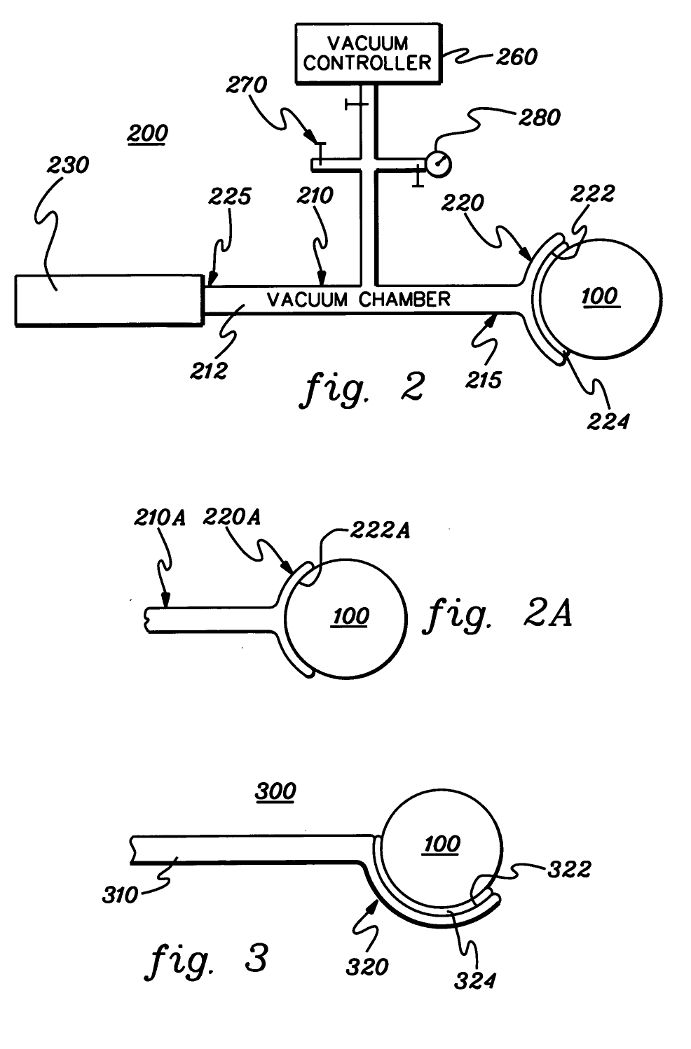

FIGS. 2-8 depict various embodiments of surgical inserter tools (in accordance with aspects of the present invention) for securely engaging, inserting and disengaging an intervertebral device, such as spacer 100 depicted in FIG. 1. In the embodiments described below, the surgical tools incorporate an insertion/extraction mechanism comprising an elongate inserter, and an engagement mechanism. The elongate inserter has proximal and distal ends, with the engagement mechanism being disposed at the distal end of the elongate inserter. The engagement mechanism includes an engagement surface configured to at least partially conform to an intervertebral device. Further, the engagement mechanism is adapted to releasably engage the intervertebral device employing either a vacuum coupling or a magnetic coupling between the engagement mechanism and the intervertebral device.

Various surgical inserter tool embodiments employing vacuum coupling are first described below with reference to FIGS. 2-5.

As shown in FIG. 2, one embodiment of a surgical inserter tool 200 employing vacuum coupling includes an insertion/extraction mechanism comprising an elongate inserter 210 having distal end 215 and proximal end 225. A handle 230, connected to proximal end 225 of elongate inserter 210, facilitates manipulation of the insertion/extraction mechanism, while an engagement mechanism 220 disposed at distal end 215 includes an engagement surface 222 configured to at least partially conform to an intervertebral device 100. By way of example, engagement mechanism 220 with engagement surface 222 are shown extending substantially symmetrical about distal end 215 of elongate inserter 210. Further, in this embodiment, a sealing gasket 224 is positioned on engagement surface 222 of the engagement mechanism. Gasket 224 is compliant and forms an air-tight seal between engagement surface 222 and a solid exterior surface portion of the intervertebral device 100 when vacuum coupling is applied to releasably engage the intervertebral device. As one example, gasket 224 may have an O-ring configuration of any desired thickness. Note that depending on the thickness of sealing gasket 224, intervertebral device 100 may or may not directly contact engagement surface 222 in this embodiment. One or more openings (not shown) are defined in engagement surface 222 within the center region of gasket 224. These openings are in communication with a vacuum chamber (or channel) 212 within elongate inserter 210.

In this example, vacuum coupling is provided by a vacuum controller (e.g., vacuum source) 260 in communication with vacuum chamber 212. By way of example, vacuum controller 260 could comprise any conventional vacuum source, including a vacuum pump, vacuum line, suction device, suction bulb, etc. Also coupled to the vacuum chamber is a release valve 270 and a vacuum meter/gage 280.

By way of example, one surgical process embodiment employing surgical inserter tool 200 includes: bringing engagement mechanism 220 in physical contact with intervertebral device 100; actuating vacuum controller 260 to initiate vacuum coupling between the engagement mechanism and the intervertebral device (note that vacuum coupling assumes that the engagement mechanism couples to a solid exterior surface portion of the intervertebral device); inserting the intervertebral device in a pre-prepared intervertebral space between vertebral members of a patient; adjusting and finalizing position of the intervertebral device in the intervertebral space; opening release valve 270 to disengage the vacuum coupling between engagement mechanism 220 and the intervertebral device 100; and thereafter, withdrawing the surgical inserter tool.

FIG. 2A partially depicts an alternate surgical inserter tool embodiment, wherein unshown components may be similar to those described above in connection with FIG. 2. In this alternate embodiment, the engagement mechanism 220A again extends substantially symmetrical about the distal end of elongate inserter 210A, and includes an engagement surface 222A that is configured to at least partially conform to an exterior surface of an intervertebral device 100 to be implanted. In this embodiment, engagement surface 222A is itself a compliant contacting surface, thus eliminating the need for a sealing gasket (such as employed in the tool embodiment of FIG. 2).

With both the tool embodiment of FIG. 2 and the tool embodiment of FIG. 2A, an air-tight seal is obtained between the engagement surface and the exterior surface of the intervertebral device. The contacting surface, whether the contoured surface 222A of FIG. 2A or the sealing gasket 224 of FIG. 2, is compliant, elastic or resilient. This surface may comprise, for example, foam, elastomer, rubber, etc. Further, the contacting surface conforms to the exterior surface of the intervertebral device. This assists in achieving good physical contact between the engagement mechanism and the intervertebral device, and an air-tight seal when vacuum coupling pressure is applied. In addition, the contacting surface, i.e., sealing gasket 224 of FIG. 2 or engagement surface 222A of FIG. 2A, may comprise a material selected to create friction between the contacting surface and the intervertebral device for secure attachment thereof with applying vacuum coupling. This material may be a thin layer of bio-compatible elastomeric material, such as silicone, polyurethane, polyolefin rubber, etc.

FIGS. 3-5 partially depict various alternate surgical inserter tool configurations, each employing vacuum coupling between the engagement mechanism and the intervertebral device. Unshown components of each surgical tool embodiment are assumed similar to those described above in connection with the embodiment of FIG. 2. Further, although each embodiment is shown with a sealing gasket forming the air-tight seal between the engagement mechanism and the intervertebral device, one or more of the embodiments could readily be adapted to a gasket-less embodiment such as depicted in FIG. 2A and described above.

Referring to FIG. 3, this surgical inserter tool 300 includes an engagement mechanism 320 at the distal end of an elongate inserter 310 having an engagement surface 322 configured to partially conform to intervertebral device 100. In this embodiment, the engagement mechanism 320 and surface 322 are offset at the distal end of the elongate inserter, resulting in a spoon-shaped tool configuration at the distal end of inserter 310. Those skilled in the art should note from this example that the engagement mechanism and engagement surface could extend in any desired asymmetrical fashion from the distal end of the elongate inserter.

In the embodiment of FIG. 3, a sealing gasket 324 is provided as a contacting surface between the engagement mechanism and the intervertebral device to form an air-tight seal therebetween, and thus allow for initiation of the vacuum coupling. Although not shown, a vacuum chamber or channel is provided, for example, within elongate inserter 310 in communication with one or more openings in surface 322 to allow for establishing of the vacuum coupling of intervertebral device 100 to engagement mechanism 320.

FIG. 4 depicts still another embodiment of a surgical inserter tool 400 employing vacuum coupling. In this embodiment, engagement mechanism 420 includes a contoured engagement surface 422 and a sealing gasket 424 to facilitate (as described above in connection with the tool embodiments of FIGS. 2 & 3) establishing of vacuum coupling of intervertebral device 100 to engagement mechanism 420. As a variation, however, elongate inserter 410 is angled or curved intermediate its distal and proximal ends. This angling or curvature may advantageously be employed when inserting an intervertebral device into the intervertebral space between vertebral members of a patient.

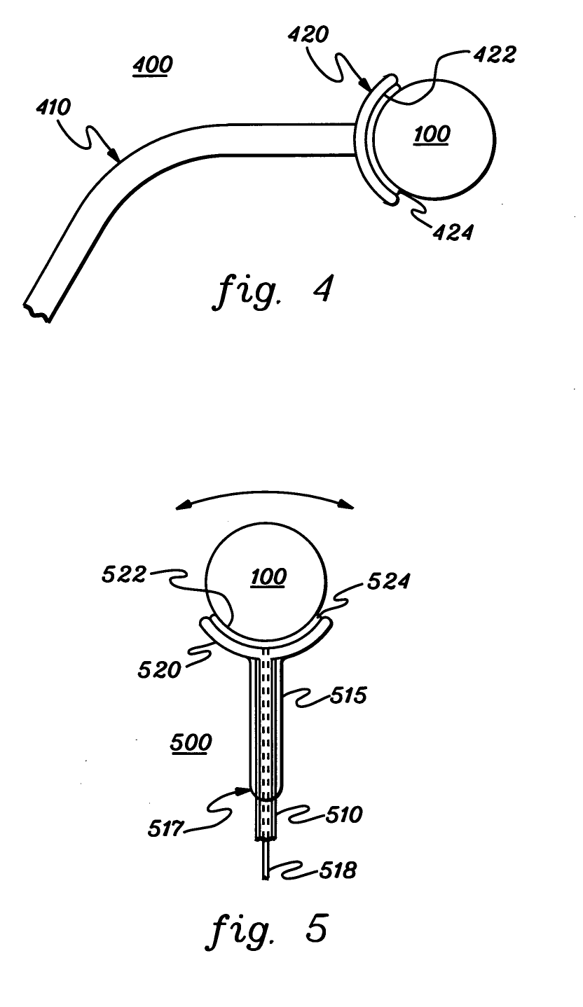

FIG. 5 depicts still another embodiment of a surgical inserter tool 500 employing vacuum coupling. Tool 500 again includes an engagement mechanism 520 similar to the engagement mechanism described above in connection with FIG. 2. Specifically, engagement mechanism 520 includes a contoured engagement surface 522 and a sealing gasket 524 that provides an air-tight seal between the engagement mechanism and an intervertebral device 100 to facilitate vacuum coupling therebetween. In this embodiment, the elongate inserter includes a first inserter portion 510 and a second inserter portion 515 pivotally joined 517 to first inserter portion 510. Passing through inserter portions 510, 515 (and in communication with one or more openings in engagement surface 522) is a flexible tubing 518, which defines the vacuum chamber (or channel). Pivotally joining the first and second inserter portions advantageously allows for tilting or pivoting of the engagement mechanism relative to the elongate inserter, which may be advantageously employed when inserting an intervertebral device between vertebral members of a patient.

Those skilled in the art will note from the above description that various aspects of the surgical inserter tool embodiments described above in connection with FIGS. 2-5 may be combined as desired. For example, the engagement mechanism and surface may be symmetrically or asymmetrically positioned relative to the distal end of the elongate inserter in any desired configuration. Further, those skilled in the art should note that the insertion/extraction mechanism described herein can be straight, curved or angled (see FIG. 4), tiltable or pivotable (see FIG. 5), rotatable, extenable, retractable, disposable, radiolucent, semi-radiolucent, etc. Further, one or more components of the insertion/extraction mechanism can be fabricated of metal, polymer, ceramic, or any combination thereof. The components may be totally disposable or partially disposable.

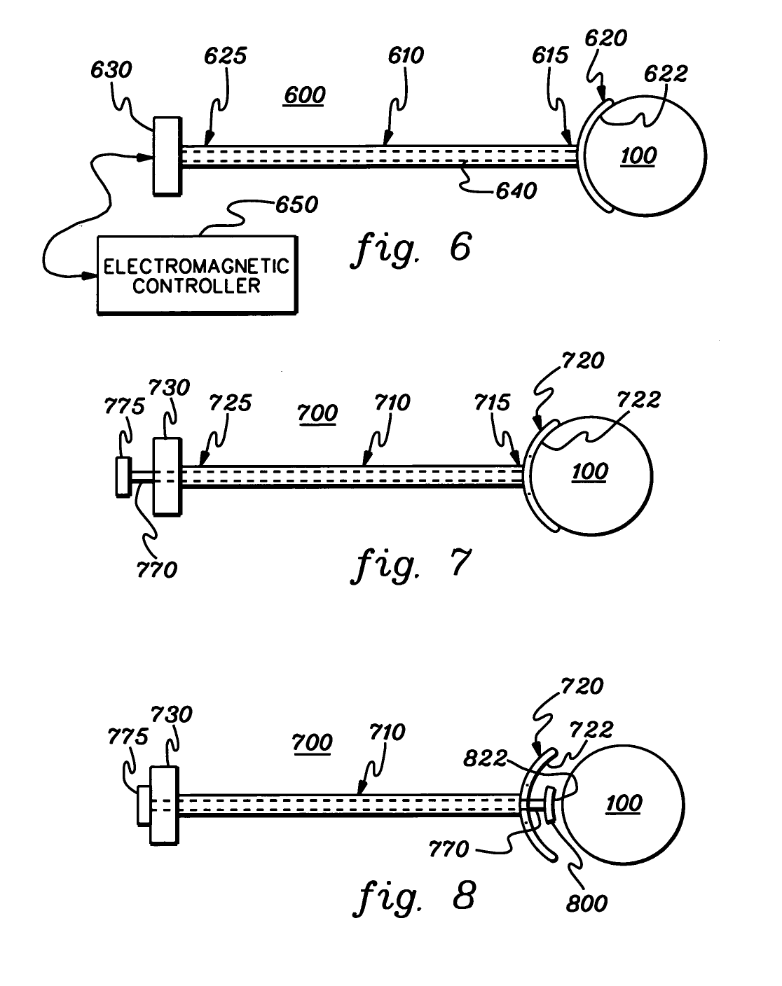

FIGS. 6-8 depict various surgical inserter tool embodiments employing magnetic coupling between the tool and an intervertebral device.

In the tool embodiment of FIG. 6, electromagnetism is employed to achieve a temporary magnetic coupling between tool 600 and intervertebral device 100. Tool 600 again includes an elongate inserter 610 with distal 615 and proximal 625 ends. An engagement mechanism 620, disposed at distal end 615 of inserter 610, includes one or more electromagnetic conductors and an engagement surface 622 configured to at least partially conform to an intervertebral device 100. In one embodiment, inserter 610 includes a metallic shaft 640 coupled to an electromagnetic controller 650. By supplying current in a well known manner, a temporary magnetic field can be achieved in the one or more electromagnetic conductors of engagement mechanism 620. Note that the tool embodiments of FIGS. 6-8 assume that intervertebral device 100 is at least partially formed of a metallic material susceptible to a magnetic attractive force generated (or existing) within engagement mechanism 620.

As a further alternative, a permanent magnetic coupling could be employed by a surgical inserter tool 700 such as shown in FIGS. 7 & 8. Tool 700 includes an elongate inserter 710 having distal 715 and proximal 725 ends. An engagement mechanism 720 is again coupled to distal end 715 of inserter 710, while a handle 730 is disposed at the proximal end 725. In this embodiment, engagement mechanism 720 is itself a permanent magnet or is configured to include one or more permanent magnets imbedded therein. Further, mechanism 720 includes an engagement surface 722 configured to at least partially conform to an exterior surface of an intervertebral device 100.

In one embodiment, elongate inserter 710 is a sleeve movably mounted to a pusher shaft 770. In this embodiment, pusher shaft 770 includes a non-magnetic pusher element 800 having a pusher surface 822 at one end, and a handle 775 at an opposite end. The inserter 710 reciprocates between an extended position (shown in FIG. 7), and a retracted position (shown in FIG. 8). In the extended position, the intervertebral device is magnetically coupled to engagement mechanism 720 of surgical inserter tool 700, while in the retracted position, the pusher element at the end of pusher shaft 770 physically separates the intervertebral device from the surgical inserter tool, interrupting the magnetic coupling therebetween. In one embodiment, pusher element surface 822 is similarly contoured with a similar radius of curvature as surface 722 of engagement mechanism 720.

As a further variation, one or more permanent magnetic couplings could be employed in a surgical inserter tool wherein the permanent magnets are disposed on a distal end of a shaft, movably mounted relative to an elongate inserter. In such an embodiment, the permanent magnet(s) can be pushed forward to be even with an engagement surface of the engagement mechanism, and thereby engage the intervertebral device, or retracted from the engagement mechanism and surface, to thereby disengage the intervertebral device from magnetic coupling with the permanent magnet(s). In this embodiment, the engagement surface is a compliant surface that remains substantially stationary and contacts the intervertebral device while engaging or disengaging the magnetic coupling is accomplished using the permanent magnet(s) on the distal end of the shaft. This alternative surgical tool configuration could alternatively be employed with electromagnetic conductors on the distal end of the shaft for active electrical control of the magnetic coupling between the surgical tool and the intervertebral device (as in the embodiment of FIG. 6).

By way of further example, any one of the above-described surgical inserter tools could be employed to position an intervertebral device in the intervertebral space between vertebral members of a patient. The surgical method includes employing a surgical tool having: an insertion/extraction mechanism including an elongate inserter and an engagement mechanism, the elongate inserter having proximal and distal ends, with the engagement mechanism being disposed at the distal end of the elongate inserter, and wherein the engagement mechanism includes an engagement surface configured to at least partially conform to an intervertebral device; and wherein the engagement mechanism is adapted to releasably engage the intervertebral device employing a vacuum coupling or a magnetic coupling between the engagement mechanism and the intervertebral device.

Employing of the surgical tool includes: employing the engagement mechanism at the distal end of the elongate inserter to releasably engage the intervertebral device using either vacuum coupling or magnetic coupling; inserting the intervertebral device between vertebral members of a patient employing the surgical tool; and detaching the intervertebral device from the engagement mechanism by disengaging the employed vacuum coupling or magnetic coupling therebtween.

As a still further example, any one of the above-described surgical inserter tools could also be employed to withdraw an intervertebral device from the intervertebral space between vertebral members of a patient. In such a case, employing of such a tool includes: inserting the engagement mechanism of the surgical tool into the patient to physically contact the intervertebral device; controllably activating the engagement mechanism to releasably engage the intervertebral device using one of vacuum coupling or magnetic coupling; and withdrawing the engagement mechanism and intervertebral device from the patient and, thereafter, detaching the intervertebral device from the engagement mechanism by disengaging the vacuum coupling or magnetic coupling therebetween.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims.

What is claimed is:

1. A surgical tool for inserting/extracting an intervertebral device, the surgical tool comprising:
an insertion/extraction mechanism comprising an elongate inserter and an engagement mechanism, the elongate inserter having proximal and distal ends, with the engagement mechanism being disposed at the distal end of the elongate inserter, and wherein the engagement mechanism includes an engagement surface configured to at least partially conform to an intervertebral device; and
wherein the engagement mechanism is adapted to releasably engage the intervertebral device employing a vacuum coupling or a magnetic coupling between the engagement mechanism and the intervertebral device.

2. The surgical tool of claim 1, wherein the engagement mechanism employs vacuum coupling to releasably engage the intervertebral device, and wherein the engagement surface is a compliant surface adapted to form an air-tight seal between the engagement mechanism and the intervertebral device when the vacuum coupling is applied.

3. The surgical tool of claim 2, wherein the insertion/extraction mechanism further comprises a vacuum chamber, and wherein the engagement surface includes at least one opening in communication with the vacuum chamber for establishing vacuum coupling between the engagement mechanism and the intervertebral device when the intervertebral device is in physical contact with the engagement surface.

4. The surgical tool of claim 3, further comprising a vacuum controller coupled to the vacuum chamber, the vacuum controller controllably establishing a vacuum pressure coupling between the engagement mechanism and the intervertebral device when the intervertebral device is in physical contact with the engagement surface.

5. The surgical tool of claim 3, wherein the elongate inserter comprises a first inserter portion and a second inserter portion pivotally mounted to each other to facilitate insertion of the intervertebral device between vertebral members of a patient or extraction of the intervertebral device from between vertebral members of a patient, and wherein the insertion/extraction mechanism further comprises a flexible tube disposed within the first and second inserter portions, the flexible tube defining at least a portion of the vacuum chamber.

6. The surgical tool of claim 2, wherein the intervertebral device comprises a solid exterior surface portion, and wherein the engagement surface is configured to at least partially conform to the solid exterior surface portion of the intervertebral device.

7. The surgical tool of claim 2, wherein the elongate inserter includes at least one bend intermediate the proximal and distal ends thereof to facilitate insertion of the intervertebral device between vertebral members of a patient or extraction of the intervertebral device from between vertebral members of a patient.

8. The surgical tool of claim 1, wherein the engagement mechanism employs vacuum coupling to releasably engage the intervertebral device, and wherein the engagement mechanism further comprises a sealing gasket positioned on the engagement surface of the engagement mechanism, the sealing gasket being compliant and forming an air-tight seal between the engagement surface and the intervertebral device when the vacuum coupling is applied to releasably engage the intervertebral device.

9. The surgical tool of claim 8, wherein the insertion/extraction mechanism further comprises a vacuum chamber, and wherein the engagement surface includes at least one opening in communication with the vacuum chamber for establishing vacuum coupling between the engagement mechanism and the intervertebral device when the intervertebral device is in physical contact with the sealing gasket on the engagement surface of the engagement mechanism.

10. The surgical tool of claim 9, further comprising a vacuum controller coupled to the vacuum chamber, the vacuum controller controllably establishing a vacuum pressure coupling between the engagement mechanism and the intervertebral device when the intervertebral device is in physical contact with the sealing gasket on the engagement surface of the engagement mechanism.

11. The surgical tool of claim 9, wherein the elongate inserter comprises a first inserter portion and a second inserter portion pivotally mounted to each other to facilitate insertion of the intervertebral device between vertebral members of a patient or extraction of the intervertebral device from between vertebral members of a patient, and wherein the insertion/extraction mechanism further comprises a flexible tube disposed within the first and second inserter portions, the flexible tube defining at least a portion of the vacuum chamber.

12. The surgical tool of claim 8, wherein the intervertebral device comprises a solid exterior surface portion, and wherein the engagement surface is configured to at least partially conform to the solid exterior surface portion of the intervertebral device.

13. The surgical tool of claim 8, wherein the elongate inserter includes at least one bend intermediate the proximal and distal ends thereof to facilitate insertion of the intervertebral device between vertebral members of a patient or extraction of the intervertebral device from between vertebral members of a patient.

14. The surgical tool of claim 1, wherein the intervertebral device comprises metal and the engagement mechanism employs electromagnetic coupling to releasably engage the intervertebral device, and wherein the surgical tool further comprises an electromagnetic controller for electrically controlling generation of a magnetic coupling force between the engagement mechanism and the intervertebral device.

15. The surgical tool of claim 1, wherein the intervertebral device comprises metal and the engagement mechanism employs magnetic coupling to releasably engage the intervertebral device, and wherein the engagement mechanism further comprises at least one permanent magnet for generating the magnetic coupling.

16. The surgical tool of claim 15, wherein the elongate inserter is hollow and the insertion/extraction mechanism further comprises a reciprocating pusher shaft disposed within and extending partially through the elongate inserter, the pusher shaft reciprocating between a retracted position and an extended position, wherein in the retracted position, the intervertebral device couples to the engagement surface of the engagement mechanism via magnetic coupling, and in the extended position, the pusher shaft detaches the intervertebral device from the engagement surface of the engagement mechanism, thereby disengaging the intervertebral device from the engagement mechanism.

17. The surgical tool of claim 16, wherein the pusher shaft includes proximal and distal ends, the distal end including a pusher member surface with a radius of curvature corresponding to a radius of curvature of the engagement surface of the engagement mechanism, and wherein the proximal end of the pusher member comprises a handle for facilitating reciprocation of the pusher shaft between the retracted and extended positions.

18. The surgical tool of claim 15, wherein the elongate inserter is hollow and the insertion/extraction mechanism further comprises a reciprocating shaft disposed within the elongate inserter, the shaft reciprocating between an extended position and a retracted position, and including a distal end comprising the at least one permanent magnet, wherein in the extended position, the distal end of the shaft couples the intervertebral device to the engagement mechanism via magnetic coupling, and in the retracted position, the magnetic coupling of the intervertebral device to the distal end of the shaft is disengaged, thereby detaching the intervertebral device from the engagement mechanism.

19. A surgical tool for inserting/extracting an intervertebral device, the surgical tool comprising:
an insertion/extraction mechanism comprising an elongate inserter and an engagement mechanism, the elongate inserter having proximal and distal ends, with the engagement mechanism being disposed at the distal end of the elongate inserter, and wherein the engagement mechanism includes an engagement surface configured to at least partially conform to an intervertebral device; and
a controller for controlling application of a coupling force between the engagement mechanism and the intervertebral device to releasably engage the intervertebral device when disposed adjacent to the engagement surface for facilitating inserting of the intervertebral device between vertebral members of a patient or extraction of the intervertebral device from between vertebral members of a patient.

20. The surgical tool of claim 19, wherein the coupling force comprises one of a vacuum coupling force or a magnetic coupling force.

21. The surgical tool of claim 20, wherein the coupling force is a vacuum coupling force, and wherein the controller comprises a vacuum controller for controllably establishing vacuum coupling pressure between the engagement mechanism and the intervertebral device.

22. The surgical tool of claim 21, wherein the engagement surface of the engagement mechanism is a compliant surface adapted to form an air-tight seal between the engagement mechanism and the intervertebral device when vacuum coupling pressure is applied to releasably engage the intervertebral device.

23. The surgical tool of claim 21, wherein the engagement mechanism further comprises a sealing gasket positioned on the engagement surface of the engagement mechanism, the sealing gasket being compliant and forming an air-tight seal between the engagement surface and the intervertebral device when vacuum coupling pressure is applied to releasably engage the intervertebral device.

24. The surgical tool of claim 20, wherein the coupling force is a magnetic coupling force, and wherein the controller comprises an electromagnetic controller for electrically controlling generation of the magnetic coupling force between the engagement mechanism and the intervertebral device.

25. A method of positioning an intervertebral device between vertebral members of a patient, the method comprising:
employing a surgical tool having:
an insertion/extraction mechanism comprising an elongate inserter and an engagement mechanism, the elongate inserter having proximal and distal ends, and the engagement mechanism being disposed at the distal end of the elongate inserter, and wherein the engagement mechanism includes an engagement surface configured to at least partially conform to an intervertebral device; and
wherein the engagement mechanism is adapted to releasably engage the intervertebral device employing a vacuum coupling or a magnetic coupling between the engagement mechanism and the intervertebral device;
wherein employing the surgical tool comprises:
employing the engagement mechanism at the distal end of the elongate inserter to releasably engage an intervertebral device using one of the vacuum coupling or magnetic coupling;
inserting the intervertebral device between vertebral members of a patient employing the surgical tool; and
detaching the intervertebral device from the engagement mechanism by disengaging the one of the vacuum coupling or magnetic coupling therebetween.

26. The method of claim 25, wherein the surgical tool further includes a controller coupled to the insertion/extraction mechanism for controllably applying the one of the vacuum coupling or magnetic coupling, and wherein the employing of the surgical tool further comprises employing the controller to generate the one of the vacuum coupling or magnetic coupling, and wherein detaching the intervertebral device from the engagement mechanism further comprises employing the controller to disengage the one of the vacuum coupling or magnetic coupling.

27. A method of withdrawing an intervertebral device from between vertebral members of a patient, the method comprising:
employing a surgical tool having:
an insertion/extraction mechanism comprising an elongate inserter and an engagement mechanism, the elongate inserter having proximal and distal ends, and the engagement mechanism being disposed at the distal end of the elongate inserter, and wherein the engagement mechanism includes an engagement surface configured to at least partially conform to an intervertebral device; and
wherein the engagement mechanism is adapted to releasably engage the intervertebral device employing a vacuum coupling or a magnetic coupling between the engagement mechanism and the intervertebral device;
wherein employing the surgical tool comprises:
inserting the engagement mechanism of the surgical tool into the patient to physically contact the intervertebral device;
controllably activating the engagement mechanism to releasably engage the intervertebral device using one of vacuum coupling or magnetic coupling; and
withdrawing the engagement mechanism and intervertebral device from the patient and, thereafter, detaching the intervertebral device from the engagement mechanism by disengaging the vacuum coupling or magnetic coupling therebetween.

28. The method of claim 27, wherein the surgical tool further includes a controller coupled to the insertion/extraction mechanism for controllably applying the one of the vacuum coupling or magnetic coupling, and wherein the controllably activating comprises employing the controller to generate the one of the vacuum coupling or magnetic coupling, and wherein detaching the intervertebral device from the engagement mechanism further comprises employing the controller to disengage the one of the vacuum coupling or magnetic coupling.
